(19) United States
(12) Patent Application Publication
(10) Pub. No.: US 2024/0216411 A1
(43) Pub. Date: Jul. 4, 2024

(54) METHODS AND COMPOSITIONS FOR TREATMENT OF CYSTIC FIBROSIS CLASS I MUTATIONS

(71) Applicant: Ann and Robert H. Lurie Children's Hospital of Chicago, Chicago, IL (US)

(72) Inventors: Hiroyuki Hirai, Chicago, IL (US); Youyang Zhao, Chicago, IL (US)

(21) Appl. No.: 18/391,200

(22) Filed: Dec. 20, 2023

Related U.S. Application Data
(60) Provisional application No. 63/476,375, filed on Dec. 20, 2022.

(51) Int. Cl. A61K 31/7056; A61K 31/121; A61K 31/19; A61K 31/352; A61K 31/404; A61K 31/4439; A61K 31/47; A61K 45/06; A61P 11/00

(52) U.S. Cl. CPC A61K 31/7056; A61K 31/121; A61K 31/19; A61K 31/352; A61K 31/404; A61K 31/4439; A61K 31/47; A61K 45/06; A61P 11/00

(57) ABSTRACT

Disclosed are methods for treating cystic fibrosis where the method comprises administering to the subject an effective amount of an inhibitor selected from the group consisting of a sodium glucose co-transporter (SGLT) inhibitor, a Na⁺/K⁺-ATPase inhibitor, a SGLT/Na³⁰/K⁺-ATPase dual inhibitor, and combinations thereof and a subeffective amount of one or more therapeutic agents selected from the group consisting of elexacaftor, tezacaftor, ivacaftor, and combinations thereof. Also disclosed are methods for treating CF characterized by a class I nonsense mutation in the CFTR gene where the method comprises administering to the subject an effective amount of a Na⁺/K⁺-ATPase inhibitor or a combination of an effective amount of a SGLT/Na⁺/K⁺-ATPase dual inhibitor and an effective amount of a Na³⁰/K⁺-ATPase inhibitor.

### CROSS-REFERENCE TO RELATED APPLICATIONS

This application claims benefit of priority to U.S. Application Ser. No. 63/476,375, filed Dec. 20, 2022, the contents of which are incorporated by reference in its entirety.

### BACKGROUND

Current cystic fibrosis transmembrane conductance regulator (CFTR) treatments include CFTR modulator therapies (e.g., TRIKAFTA) which treat cystic fibrosis characterized by class II, class III, class IV, and class VI mutations. However, there is no effective therapy for treating patients having cystic fibrosis characterized by class I or class II CFTR nonsense mutation. For example, TRIKAFTA therapy causes serious adverse reactions including hepatic injury and hypersensitivity.

As such, there exists a need for methods and compositions for treating cystic fibrosis characterized by class I or class II CFTR nonsense mutation that have a better safety profile.

### SUMMARY

Disclosed are methods for treating cystic fibrosis (CF). The disclosed methods may comprise administering to a subject an effective amount of an inhibitor selected from the group consisting of a sodium glucose co-transporter (SGLT) inhibitor, a Na⁺/K⁺-ATPase inhibitor, a SGLT/Na⁺/K⁺-ATPase dual inhibitor, and combinations thereof and a subeffective amount of one or more therapeutic agents selected from the group consisting of elexacaftor, tezacaftor, ivacaftor, and combinations thereof.

Another aspect of the invention provides for methods for treating cystic fibrosis (CF). The disclosed methods may comprise administering to a subject an effective amount of one or more inhibitors selected from the group consisting of phlorizin, T-1095, licogliflozin, trilobatin, sergliflozin, remogliflozin, ipragliflozin, luseogliflozin, tofogliflozin, empagliflozin, canagliflozin, dapagliflozin, KGA-2727, LX2761, LX276123, TP043883625, SGL5213, SGLT inhibitor 1, mizagliflozin, phloretin, pyruvic acid, acevaltrate, ouabain, bufalin, istaroxime, biacetyl monoxime, rostafuroxin, gitoxin, oleandrin, deslanoside, chlorpropamide, periplocin, digoxin, rostafuroxin; and a subeffective amount of one or more therapeutic agents selected from the group consisting of elexacaftor, tezacaftor, ivacaftor, and combinations thereof.

Another aspect of the invention provides for methods for treating cystic fibrosis (CF) characterized by a class I nonsense mutation in the cystic fibrosis transmembrane conductance regulator (CFTR) gene in a subject in need thereof. The method may comprise administering to the subject an effective amount of a Na⁺/K⁺-ATPase inhibitor. The method may comprise administering to the subject an effective amount of a SGLT/Na⁺/K⁺-ATPase dual inhibitor and an effective amount of a Na⁺/K⁺-ATPase inhibitor.

Another aspect of the invention provides for methods for treating cystic fibrosis (CF) characterized by a class I nonsense mutation in the cystic fibrosis transmembrane conductance regulator (CFTR) gene in a subject in need thereof. The method may comprise administering to the subject an effective amount of one or more inhibitors selected from the group consisting of phlorizin, T-1095, licogliflozin, trilobatin, sergliflozin, remogliflozin, ipragliflozin, luseogliflozin, tofogliflozin, empagliflozin, canagliflozin, dapagliflozin, KGA-2727, LX2761, LX276123, TP043883625, SGL5213, SGLT inhibitor 1, mizagliflozin, and phloretin; and an effective amount of one or more inhibitors selected from the group consisting of pyruvic acid, acevaltrate, ouabain, bufalin, istaroxime, biacetyl monoxime, rostafuroxin, gitoxin, oleandrin, deslanoside, chlorpropamide, periplocin, digoxin, and rostafuroxin.

### BRIEF DESCRIPTION OF THE DRAWINGS

FIGS. 1A and 1B show generation of Proximal Lung Organoids from Cystic Fibrosis patient iPSCs carrying Class I or II mutations. FIG. 1A demonstrates representative immunofluorescence staining for p63 and EpCAM in proximal Lung Organoids derived from iPSCs with or without Class I or II mutations of either dF508/dF508, W1282X/W1282X or G542X/G542X on Day 22. Nuclei were counterstained with DAPI (blue). Scale bars, 100 μm. FIG. 1B shows Western blot results from cWT/cWT, dF508/dF508, W1282X/W1282X and dF/G542X/G542X HLOs.

FIGS. 2A-2C show that combination of SGLT1-selective inhibitor Mizagliflozin and low dose of TRIKAFTA synergistically restores HLO swelling of Class II CFTR dF508/dF508 mutation. FIG. 2A shows quantification of organoid swelling of HLOs with Class II dF508/dF508 mutation treated with TRIKAFTA (100 nM VX-770 and 3 μM VX-445 and 3 μM VX-661) (n=10). FIG. 2B shows quantification of organoid swelling of HLOs with Class II dF508/dF508 mutation treated with 50-fold diluted TRIKAFTA (2 nM VX-770 and 0.06 μM VX-445 and 0.06 μM VX-661) (n=10). FIG. 2C shows quantification of organoid swelling of HLOs with Class II dF508/dF508 mutation treated with 125-fold diluted TRIKAFTA (0.8 nM VX-770 and 0.024 μM VX-445 and 0.024 μM VX-661), with or without Mizagliflozin (10, 100, 1000 μM) (n=20). *P<0.05; **P<0.01; ***P<0.005.

FIG. 3 shows combination of low dose of TRIKAFTA and SGLT and Na⁺/K⁺-ATPase dual inhibitor phlorizin (also a non-selective SGLT inhibitor) restores HLO swelling of Class II CFTR dF508/dF508 mutation. Quantification of organoid swelling of HLOs with Class II dF508/dF508 mutation treated with 125-fold diluted TRIKAFTA (0.8 nM VX-770 and 0.024 μM VX-445 and 0.024 μM VX-661), without or with either 20 μM Phlorizin or 20 μM Sotagliflozin (a SGLT2-selective inhibitor). (n=17). ***P<0.005.

FIGS. 4A-4B show Na⁺/K⁺-ATPase inhibitors pyruvic acid and Acevaltrate markedly restores HLO swelling of Class I CFTR mutations. FIG. 4A shows quantification of organoid swelling of W1282X/W1282X HLOs treated with pyruvic acid (1, 10, 100, 1000 μM). (n=10). FIG. 4B demonstrates quantification of organoid swelling of W1282X/W1282X HLOs treated with Acevaltrate (5, 20, 50 μM). (n=20). *P<0.05; ***P<0.005.

FIG. 5 demonstrates combination of Na⁺/K⁺-ATPase inhibitor Ouabain and SGLT1/Na⁺/K⁺-ATPase dual inhibitor Phlorizin synergistically restores HLO swelling of Class I CFTR W1282X/W1282X mutation. Quantification of organoid swelling of HLOs with Class I W1282X/W1282X mutation treated with or without Ouabain (100 nM) or Phlorizin (20 μM) or Ouabain and Phlorizin combination. (n=13). *P<0.05; ***P<0.005.

### DETAILED DESCRIPTION

The present invention is described herein using several definitions, as set forth below and throughout the application.

Unless otherwise specified or indicated by context, the terms “a”, “an”, and “the” mean “one or more.” For example, “a sodium glucose co-transporter (SGLT) inhibitor” should be interpreted to mean “one or more sodium glucose co-transporter (SGLT) inhibitors.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Disclosed are methods for treating cystic fibrosis (CF) comprising administering to a subject an effective amount of an inhibitor selected from the group consisting of a sodium glucose co-transporter (SGLT) inhibitor, a Na⁺/K⁺-ATPase inhibitor, a SGLT/Na⁺/K⁺-ATPase dual inhibitor, and combinations thereof and a subeffective amount of one or more therapeutic agents selected from the group consisting of elexacaftor, tezacaftor, ivacaftor, and combinations thereof.

In some embodiments, the CF to be treated is characterized by a class I, II, III, IV, or VI mutation in the cystic fibrosis transmembrane conductance regulator (CFTR) gene. In some embodiments, the CF to be treated is characterized by a class I mutation in the CFTR gene. In some embodiments, the CF to be treated is characterized by a class II mutation in the CFTR gene.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disorder. As such, the methods disclosed herein encompass both therapeutic and prophylactic administration.

As used herein, the term “subject in need thereof” refers to a subject having or at risk for developing cystic fibrosis, including cystic fibrosis characterized by a class I nonsense mutation or a class II mutation in the cystic fibrosis transmembrane conductance regulator (CFTR) gene. A “subject in need thereof” may include a human or non-human subject (e.g., anon-human mammal).

As used herein, the term “effective amount” refers to the amount or dose of the compound, upon single or multiple dose administration to the subject, which provides the desired effect in the subject under diagnosis or treatment. The disclosed methods may include administering an effective amount of the disclosed inhibitors (e.g., as present in a pharmaceutical composition) for treating cystic fibrosis (CF) characterized by a class I nonsense mutation in the cystic fibrosis transmembrane conductance regulator (CFTR) gene.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual patient; the particular inhibitor administered; the mode of administration; the bioavailability characteristics of the preparation administered; the dose regimen selected; the use of concomitant medication; and other relevant circumstances.

When administered in combination with the inhibitor, the effective concentration of the one or more therapeutic agent to elicit a particular biological response is a subeffective amount, i.e., less than the effective concentration of each agent when administered alone, thereby allowing a reduction in the dose of one or more of the agents relative to the dose that would be needed if the agent was administered as a single agent. The effects of multiple agents may, but need not be, additive or synergistic.

As used herein, the “subeffective amount” may be a dose from one five thousandth ( 1/5000) to one half (½) of the dose of the co-administered agent (e.g., a SGLT inhibitor, a Na⁺/K⁺-ATPase inhibitor, a SGLT/Na/KW-ATPase dual inhibitor, etc.) in the methods as disclosed herein. In some embodiments, the subeffective amount of the agent may be a dose from 1/1000 to ½, or from 1/500 to ½, or from 1/450 to ½, from 1/400 to ½, from 1/350 to ½, from 1/300 to ½, from 1/250 to ½, from 1/200 to ½, from 1/150 to ½, from 1/125 to ½, from 1/50 to ½, from 1/20 to ½, from 1/10 to ½, from ⅕ to ½, or from ⅓ to ½ of the dose of the co-administered agent. In some embodiments, the subeffective amount of the agent may be a dose from 1/5000 to ⅕, or from 1/5000 to 1/10, or from 1/5000 to 1/20, from 1/5000 to 1/50, from 1/5000 to 1/125, from 1/5000 to 1/150, from 1/5000 to 1/200, from 1/5000 to 1/250, from 1/5000 to 1/300, from 1/5000 to 1/350, from 1/5000 to 1/400, from 1/5000 to 1/450, from 1/5000 to 1/500, or from 1/5000 to 1/1000 of the dose of the co-administered agent.

In some embodiments, a daily dose of the disclosed inhibitors may contain from about 0.01 mg/kg to about 100 mg/kg (such as from about 0.05 mg/kg to about 50 mg/kg and/or from about 0.1 mg/kg to about 25 mg/kg) of each inhibitor used in the present method of treatment. The dose may be administered under any suitable regimen (e.g., weekly, daily, twice daily).

As used herein, an “inhibitor” refers to a compound that inhibits the activity of a protein of interest, such as a SGLT (e.g., SGLT1 and/or SGLT2) or Na⁺/K⁺-ATPase protein. Suitably, the inhibitor may have a IC₅₀ of less than 1000 nM, less than 500 nM, less than 200 nM, less than 100 nM, less than 50 nM, less than 40 nM, less than 30 nM, less than 20 nM, less than 10 nM, less than 5 nM, less than 2.5 nM, less than 1 nM, less than 0.5 nM, or less than 0.1 nM for the protein of interest.

In some embodiments, the inhibitor used in the methods as described herein is a SGLT/Na⁺/K⁺-ATPase dual inhibitor. As used herein, the term “SGLT/Na⁺/K⁺-ATPase dual inhibitor” refers to an inhibitor that inhibits both SGLT protein (e.g., SGLT1 and/or SGLT2) and Na⁺/K⁺-ATPase. In some embodiments, the SGLT/Na⁺/K⁺-ATPase dual inhibitor is a SGLT1/Na⁺/K⁺-ATPase dual inhibitor.

In some embodiments, the SGLT/Na⁺/K⁺-ATPase dual inhibitor is selected from the group consisting of phlorizin, T-1095, licogliflozin, trilobatin, sergliflozin, remogliflozin, ipragliflozin, luseogliflozin, tofogliflozin, empagliflozin, canagliflozin, dapagliflozin, and analogs thereof.

As used herein the term “analog” refers to compounds having similar physical, chemical, biochemical, or pharmacological properties, which include structural analogs and/or functional analogs. The term “structural analog” or “chemical analog” refers to a compound having a structure similar to that of another compound, but differing with respect to one or more structural moieties (e.g., one or more atoms, functional groups, or substructures). A structural analog of a compound can theoretically be formed from that compound after one or more chemical reactions. The term “functional analog” may include compounds that are not necessarily structural analogs with a similar chemical structure. An example of pharmacological functional analogs are morphine, heroine, and fentanyl, which have the same mechanism of action, but fentanyl is structurally different from the other two.

The term “phlorizin” (also known as phloridzin) refers to a compound having a formula C₂₁H₂₄O₁₀ and an IUPAC name (1-(2,4-dihydroxy-6-{[(2S,3R,4S,5S,6R)-3,4,5-trihydroxy-6-(hydroxymethyl)oxan-2-yl]oxy}phenyl)-3-(4-hydroxyphenyl)propan-1-one). The chemical structure of phlorizin is shown below:

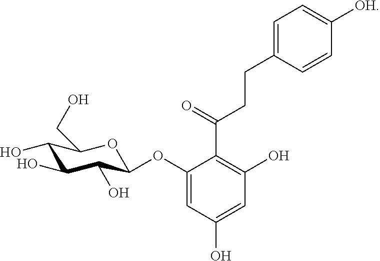

Phlorizin is a glucoside of phloretin, a dihydrochalcone, found primarily in unripe Malus (apple) root bark of apple. Phlorizin is an inhibitor of SGLT1 and SGLT2 (i.e., non-selective SGLT inhibitor) because it competes with D-glucose for binding to the carrier. Phlorizin is also an inhibitor of Na⁺/K⁺-ATPase. Examples of phlorizin analogs include, but are not limited to, trilobatin, T-1095, sergliflozin, remogliflozin, licogoliflozin, ipragliflozin, luseogliflozin, tofogliflozin, empagliflozin, canagliflozin, and dapagliflozin.

The term “T-1095” refers to a compound having a chemical formula C₂₆H₂₈O₁₁, and a chemical name ((2R,3S,4S,5R,6S)-6-(2-(3-(benzofuran-5-yl)propanoyl)-3-hydroxy-5-methylphenoxy)-3,4,5-trihydroxytetrahydro-2H-pyran-2-yl)methyl methyl carbonate. The structure of T-1095 is shown below:

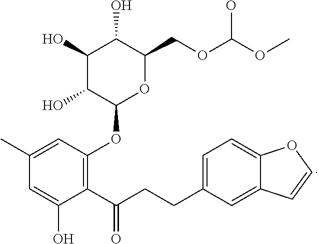 [SMILES: COC(O)OC[C@H]1O[C@@H](Oc2cc(C)cc(O)c2C(=O)CCc2ccc3occc3c2)[C@H](O)[C@@H](O)[C@@H]1O]

The term “licogliflozin” refers to a compound having a chemical formula C₂₃H₂₈O₇ and an IUPAC name (2S,3R 4R 5S6R)-2-[3-(2,3-dihydro-1,4-benzodioxin-6-ylmethyl)-4-ethylphenyl]-6-(hydroxymethyl)oxane-3,4,5-triol. The structure of licogliflozin is shown below:

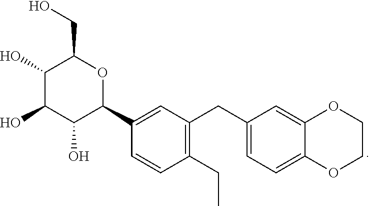 [SMILES: CCc1ccc([C@@H]2O[C@H](CO)[C@@H](O)[C@H](O)[C@H]2O)cc1Cc1ccc2c(c1)OCCO2]

In some embodiments, the inhibitor used in the methods as described herein is a SGLT inhibitor. The term “SGLT inhibitor” refers to SGLT1-selective inhibitor, SGLT-2 selective inhibitor, and non-selective SGLT inhibitor. In some embodiments, the SGLT inhibitor is a SGLT1-selective inhibitor.

As used herein, the term “SGLT1-selective inhibitor” refers to an inhibitor that has a greater inhibitory affect against SGLT1 than SGLT2. In some embodiments, the SGLT1-selective inhibitor may have an IC₅₀ for SGLT2 that is at least 5, 10, 20, 50, 100, 200, 500, 1000, 2000, 5000, or 10,000 times greater than for SGLT1.

In some embodiments, the SGLT1-selective inhibitor is selected from the group consisting of KGA-2727, LX2761, LX276123, TP043883625, SGL5213, SGLT inhibitor 1, mizagliflozin, and analogs thereof. In some embodiments, the SGLT1-selective inhibitor is mizagliflozin or analogs thereof.

The term “SGLT inhibitor 1” refers to a compound having a chemical formula C₂₄H₂₇FO₈ and a structure as shown below:

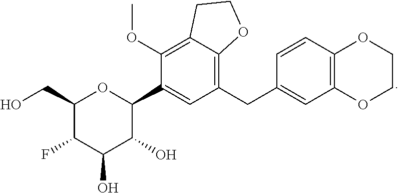 [SMILES: COc1c([C@@H]2O[C@H](CO)[C@@H](F)[C@H](O)[C@H]2O)cc(Cc2ccc3c(c2)OCCO3)c2c1CCO2]

SGLT inhibitor 1 is a potent dual inhibitor of sodium glucose co-transporter proteins (SGLTs), inhibits hSGLT1 and hSGLT2 with IC_{50S}of 43 nM and 9 nM, respectively.

The term “mizagliflozin” refers to a compound having a chemical formula C₂₈H₄₄N₄O₈ and an IUPAC name 2,2-dimethyl-3-[3-[3-methyl-4-[[5-propan-2-yl-3-[(2S,3R,4S,5,6R)-3,4,5-trihydroxy-6-(hydroxymethyl)oxan-2-yl]oxy-1H-pyrazol-4-yl]methyl]phenoxy]propylamino]propanamide. The structure of mizagliflozin is shown below:

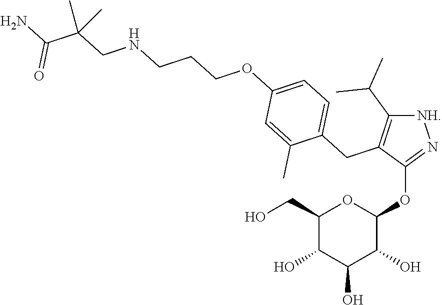 [SMILES: Cc1cc(OCCCNCC(C)(C)C(N)=O)ccc1Cc1c(O[C@@H]2O[C@H](CO)[C@@H](O)[C@H](O)[C@H]2O)n[nH]c1C(C)C]

Mizagliflozin is a potent, orally active and selective SGLT1 inhibitor, with a Kᵢ of 27 nM for human SGLT1. Mizagliflozin displays 303-fold selectivity over SGLT2. Mizagliflozin is used as an antidiabetic drug that can modify postprandial blood glucose excursion. Mizagliflozin also exhibits potential in the amelioration of chronic constipation. Examples of mizagliflozin analogs include, but are not limited to, LX276123 and TP043883625.

The term “KGA-2727” refers to a compound having a chemical formula C₂₆H₄₀N₄O₈ and a chemical name 3-((3-(4-((5-isopropyl-3-(((2S,3R,4S,5S,6R)-3,4,5-trihydroxy-6-(hydroxymethyl)tetrahydro-2H-pyran-2-yl)oxy)-1H-pyrazol-4-yl)methyl)-3-methylphenoxy)propyl)amino)propanamide. The structure of KGA-2727 is shown below:

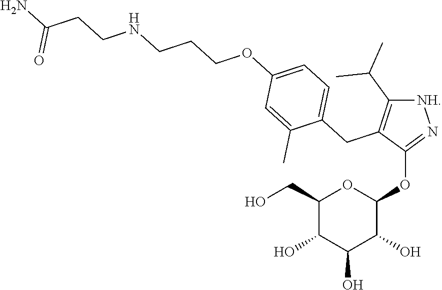 [SMILES: Cc1cc(OCCCNCCC(N)=O)ccc1Cc1c(O[C@@H]2O[C@H](CO)[C@@H](O)[C@H](O)[C@H]2O)n[nH]c1C(C)C]

KGA-2727 is a selective, high-affinity and orally active SGLT1 inhibitor with K_{iS} of 97.4 nM for human SGLT1.

The term “SGL5213” refers to a compound having a chemical formula C₂₆H₄₀N₄O₈ and an IUPAC name (E)-N-(1-((2-(dimethylamino)ethyl)amino)-2-methyl-1-oxopropan-2-yl)-4-(4-(2-isopropyl-4-methoxy-5-((2S,3R,4R,5S,6R)-3,4,5-trihydroxy-6-(hydroxymethyl)tetrahydro-2H-pyran-2-yl)benzyl)phenyl)-2,2-dimethylbut-3-enamide. The structure of SGL5213 is shown below:

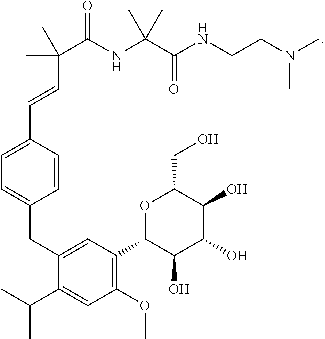 [SMILES: COc1cc(C(C)C)c(Cc2ccc(/C=C/C(C)(C)C(=O)NC(C)(C)C(=O)NCCN(C)C)cc2)cc1[C@@H]1O[C@H](CO)[C@@H](O)[C@H](O)[C@H]1O]

SGL5213 is a potent, orally active and low-absorbable sodium-dependent glucose cotransporter 1 (SGLT1) inhibitor, with IC₅₀ values of 29 nM and 20 nM for hSGLT1 and hSGLT2, respectively.

The term “LX2761” refers to a compound having a chemical formula C₃₂H₄₇N₃O₆S and an IUPAC name N-(1-((2-(Dimethylamino)ethyl)amino)-2-methyl-1-oxopropan-2-yl)-4-(4-(2-methyl-5-((2S,3R,4R,5S,6R)-3,4,5-trihydroxy-6-(methylthio)tetrahydro-2H-pyran-2-yl)benzyl)phenyl)-butanamide. The structure of LX2761 is shown below:

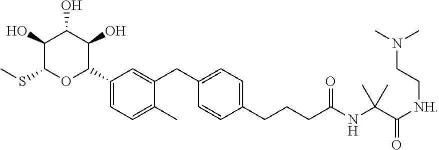

LX2761 is a potent inhibitor against SGLT1 and SGLT2 in vitro with IC_{50S} of 2.2 nM and 2.7 nM for hSGLT1 and hSGLT2, but displays specific SGLT1 inhibition in the gastrointestinal tract.

In some embodiments, the SGLT inhibitor used in the methods as described herein is a non-selective SGLT inhibitor.

As used herein, the term “non-selective SGLT inhibitor” refers to an inhibitor that inhibits both SGLT1 and SGLT2 proteins. The selectivity of an inhibitor for SGLT1 or SGLT2 protein may be measured by the value Kᵢ, which is the dissociation constant describing the binding affinity between the inhibitor and the enzyme, or the IC₅₀. In some embodiments, a non-selective SGLT inhibitor has a similar (less than 5-fold difference in) selectivity for SGLT1 and SGLT2 proteins or an approximately equal (50/50) selectivity for SGLT1 and SGLT2 proteins.

In some embodiments, the non-selective SGLT inhibitor is phloretin or analogs thereof, and any combination thereof.

The term “phloretin” refers to a compound having a chemical formula C₁₅H₁₄O₅ and an IUPAC name 3-(4-hydroxyphenyl)-1-(2,4,6-trihydroxyphenyl)propan-1-one. The structure of phloretin is shown below:

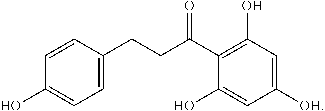 [SMILES: O=C(CCc1ccc(O)cc1)c1c(O)cc(O)cc1O]

Phloretin is a flavonoid extracted from Malus pumila Mill. and has anti-inflammatory activities. Phloretin is a specific, competitive and orally active inhibitor of sodium/glucose cotransporter SGLT1/2.

Disclosed also include a method of treating CF characterized by a class I nonsense mutation in the cystic fibrosis transmembrane conductance regulator (CFTR) gene in a subject in need thereof. The method may comprise administering to the subject an effective amount of a Na⁺/K⁺-ATPase inhibitor.

In some embodiments, the Na⁺/K⁺-ATPase inhibitor is selected from the group consisting of pyruvic acid, acevaltrate, ouabain, bufalin, istaroxime, biacetyl monoxime, rostafuroxin, gitoxin, oleandrin, deslanoside, chlorpropamide, periplocin, digoxin, rostafuroxin, analogs thereof, and any combination thereof. In some embodiments, the Na⁺/K⁺-ATPase inhibitor is selected from the group consisting of pyruvic acid, acevaltrate, and analogs thereof.

The term “ouabain” refers to a compound having a chemical formula C₂₉H₄₄O₁₂ and a chemical name 3-[(1R,3S,5S,8R,9S,10R,11R,13R,14S,17R)-1,5,11,14-tetrahydroxy-10-(hydroxymethyl)-13-methyl-3-[(2R,3R,4R,5R,6S)-3,4,5-trihydroxy-6-methyloxan-2-yl]oxy-2,3,4,6,7,8,9,11,12,15,16,17-dodecahydro-1H-cyclopenta[a]phenanthren-17-yl]-2H-furan-5-one. The structure of ouabain is shown below:

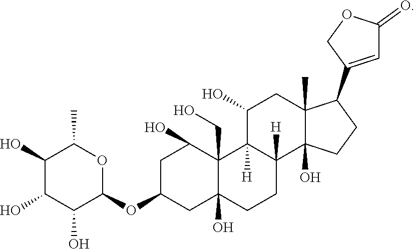

Ouabain is a cardiac glycoside that acts by inhibiting the Na⁺/K⁺-ATPase sodium-potassium ion pump (but it is not selective). Intravenous ouabain has a long history in the treatment of heart failure, and some continue to advocate its use intravenously and orally in angina pectoris and myocardial infarction. The trade name of ouabain is Strodival.

The term “bufalin” refers to a compound having a chemical formula C₂₄H₃₄O₄ and a chemical name 5-[(3S,5R,8R,9S,10S,13R,14S,17R)-3,14-dihydroxy-10,13-dimethyl-1,2,3,4,5,6,7,8,9,11,12,15,16,17-tetradecahydrocyclopenta[a]phenanthren-17-yl]pyran-2-one. The structure of bufalin is shown below:

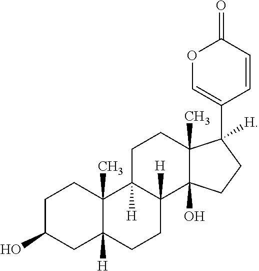

The term “istaroxime” refers to a compound having a chemical formula C₂₁H₃₂N₂O₃ and a chemical name (3E,5S,8R,9S,10R,13S,14S)-3-(2-aminoethoxyimino)-10,13-dimethyl-1,2,4,5,7,8,9,11,12,14,15,16-dodecahydrocyclopenta[a]phenanthrene-6,17-dione. The structure of istaroxime is shown below:

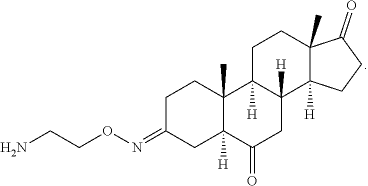

The term “biacetyl monoxime” refers to a compound having a chemical formula C₄H₇NO₂. The structure of biacetyl monoxime is shown below:

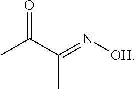

The term “rostafuroxin” refers to a compound having a chemical formula C₂₃H₃₄O₄ and a chemical name (3β,5β,14β)-21,23-epoxy-24-norchola-20,22-diene-3,14,17-triol. The structure of rostafuroxin is shown below:

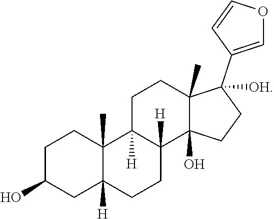

The term “gitoxin” refers to a compound having a chemical formula C₄₁H₆₄O₁₄ and an IUPAC name 3-[(3S,5R,8R,9S,10S,13R,14S,16S,17R)-3-[(2R,4S,5S,6R)-5-[(2S,4S,5S,6R)-5-[(2S,4S,5S,6R)-4,5-dihydroxy-6-methyloxan-2-yl]oxy-4-hydroxy-6-methyloxan-2-yl]oxy-4-hydroxy-6-methyloxan-2-yl]oxy-14,16-dihydroxy-10,13-dimethyl-1,2,3,4,5,6,7,8,9,11,12,15,16,17-tetradecahydrocyclopenta[a]phenanthren-17-yl]-2H-furan-5-one. The structure of gitoxin is shown below:

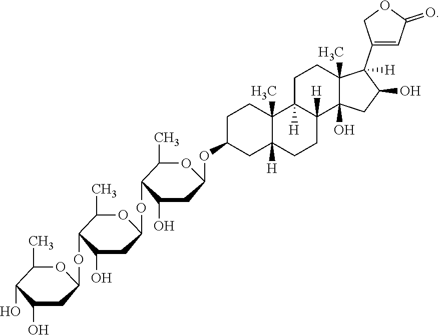

The term “oleandrin” refers to a compound having a chemical formula C₃₂H₄₈O₉ and an IUPAC name [(3S,5R,8R,9S,10S13R,14S,16S17R)-14-hydroxy-3-[(2R,4S,5S,6S)-5-hydroxy-4-methoxy-6-methyloxan-2-yl]oxy-10,13-dimethyl-17-(5-oxo-2H-furan-3-yl)-1,2,3,4,5,6,7,8,9,11,12,15,16,17-tetradecahydrocyclopenta[a]phenanthren-16-yl] acetate. The structure of gitoxin is shown below.

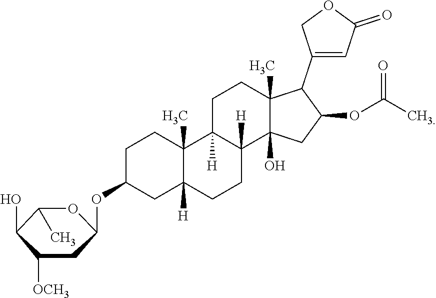

The term “deslanoside” refers to a compound having a chemical formula C₄₇H₇₄O1₉ and an IUPAC name 3-[(3S,5R,8R,9S,10S,12R,13S,14S,17R)-12,14-dihydroxy-3-[(2R,4S,5S,6R)-4-hydroxy-5-[(2S,4S,5S,6R)-4-hydroxy-5-[(2S,4S,5S,6R)-4-hydroxy-6-methyl-5-[(2S,3R,4S,5S,6R)-3,4,5-trihydroxy-6-(hydroxymethyl)oxan-2-yl]oxyoxan-2-yl]oxy-6-methyloxan-2-yl]oxy-6-methyloxan-2-yl]oxy-10,13-dimethyl-1,2,3,4,5,6,7,8,9,11,12,15,16,17-tetradecahydrocyclopenta[a]phenanthren-17-yl]-2H-furan-5-one. The structure of deslanoside is shown below:

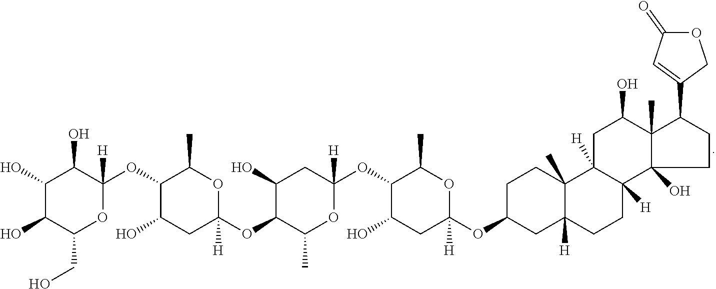 [SMILES: C[C@H]1O[C@@H](O[C@H]2CC[C@@]3(C)[C@H](CC[C@@H]4[C@@H]3C[C@@H](O)[C@]3(C)[C@@H](C5=CC(=O)OC5)CC[C@]43O)C2)C[C@H](O)[C@@H]1O[C@H]1C[C@H](O)[C@H](O[C@H]2C[C@H](O)[C@H](O[C@@H]3O[C@H](CO)[C@@H](O)[C@H](O)[C@H]3O)[C@@H](C)O2)[C@@H](C)O1]

The term “chlorpropamide” refers to a compound having a chemical formula C₁₀H₁₃C1N₂O₃S and a chemical name 4-chloro-N-(propylcarbamoyl)benzenesulfonamide. The structure of chloropropamide is shown below:

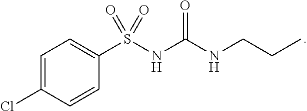 [SMILES: CCCNC(=O)NS(=O)(=O)c1ccc(Cl)cc1]

Chlorpropamide inhibits Na⁺/K⁺-ATPase. Chlorpropamide is also a drug in the sulfonylurea class used to treat non-insulin-dependent diabetes mellitus type 2. It is a long-acting first-generation sulfonylurea which causes relatively long episodes of hypoglycemia; gliclazide or tolbutamide are shorter-acting sulfonylureas. Trade names include Abemide and diabinese.

The term “periplocin” refers to a compound having a formula C₃₆H₅₆O₁₃ and a chemical name 3-[(3S,5S,8R,9S,10R,13R,14S,17R)-5,14-dihydroxy-3-[(2R,4S,5R,6R)-4-methoxy-6-methyl-5-[(2S,3R,4S,5S,6R)-3,4,5-trihydroxy-6-(hydroxymethyl)oxan-2-yl]oxyoxan-2-yl]oxy-10,13-dimethyl-2,3,4,6,7,8,9,11,12,15,16,17-dodecahydro-1H-cyclopenta[a]phenanthren-17-yl]-2H-furan-5-one. The structure of marinobufogenin is shown below:

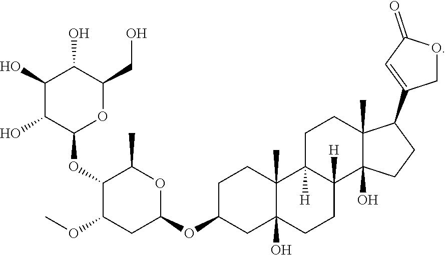

In some embodiments, the Na⁺/K⁺-ATPase inhibitor is ouabain or analogs thereof.

In some embodiments, the Na⁺/K⁺-ATPase inhibitor is chlorpropamide or analogs thereof.

In some embodiments, the method comprises administering a subeffective amount of one or more therapeutic agents that are elexacaftor, tezacaftor, and ivacaftor. In some embodiments, the combination of elexacaftor, tezacaftor, and ivacaftor is commercially available and sold under the brand name TRIKAFTA@.

In some embodiments, the methods as described herein may employ combination therapies, which means that a subject is administered at least two different active agents. For example, the active agents may be combined and administered in a single dosage form, may be administered as separate dosage forms at the same time, or may be administered as separate dosage forms that are administered alternately or sequentially on the same or separate days. In some embodiments, the active agents are combined and administered in a single dosage form. In some embodiments, the active agents are administered in separate dosage forms. In some embodiments, such combination therapies utilize lower dosages of the conventional therapeutics, thus avoiding possible toxicity and adverse side effects incurred when those agents are used as monotherapies.

The agents, whether administered alone or in combination, may be administered multiple times, and if administered as a combination, may be administered simultaneously or not, and on the same schedule or not. By way of example, a therapeutic composition may be administered one or more times per day, one or more times per week, one or more times per month, or as often as a doctor prescribes.

In some embodiments, when administered in combination, the two or more agents can have a synergistic effect. As used herein, the terms “synergy” or “synergistic” refers to circumstances under which the biological activity of a combination of an agent and at least one additional therapeutic agent is greater than the sum of the biological activities of the respective agents when administered individually.

In some embodiments, the methods or pharmaceutical composition comprises the use of a SGLT1-selective inhibitor and one or more therapeutic agents selected from the group consisting of elexacaftor, tezacaftor, ivacaftor, and combinations thereof.

In some embodiments, the methods or pharmaceutical composition comprises the use of a non-selective SGLT inhibitor and one or more therapeutic agents selected from the group consisting of elexacaftor, tezacaftor, ivacaftor, and combinations thereof.

In some embodiments, the methods or pharmaceutical composition comprises the use of a SGLT/Na⁺/K⁺-ATPase dual inhibitor and one or more therapeutic agents selected from the group consisting of elexacaftor, tezacaftor, ivacaftor, and combinations thereof.

In some embodiments, the methods or pharmaceutical composition comprises the use of a Na⁺/K⁺-ATPase inhibitor and one or more therapeutic agents selected from the group consisting of elexacaftor, tezacaftor, ivacaftor, and combinations thereof.

In some embodiments, the methods or pharmaceutical compositions further comprise the use of a second therapeutic agent selected from the group consisting of a CFTR modulator, a CFTR amplifier, and combinations thereof.

As used herein, the term “CFTR modulator” refers to CFTR potentiators and/or CFTR correctors. Examples of CFTR potentiators include, but are not limited to, CTP-656, NVS-QBW251, FD1860293, and N-(3-carbamoyl-5,5,7,7-tetramethyl-4,7-dihydro-5H-thieno[2,3-c]pyran-2-yl)-1H-pyrazole-5-carboxamide.

Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, and WO2013038390; and U.S. application Ser. Nos. 14/271,080 and 14/451,619.

Examples of correctors include Lumacaftor (VX-809), VX-983, GLPG2222, GLPG2665, GLPG2737, VX-152, VX-440, FDL169, FDL304, FD2052160, and FD2035659. Examples of correctors are also disclosed in US20160095858A1, and U.S. application Ser. Nos. 14/925,649 and 14/926,727.

As used herein, the term “CFTR amplifier” refers to therapeutic agents that enhance the effect of known CFTR modulators, such as potentiators and correctors. An example of a CFTR amplifier is PTI130. Examples of amplifiers are also disclosed in publications WO2015138909 and WO2015138934.

In some embodiments, the second therapeutic agent included in the methods as described herein is a CFTR modulator selected from the group consisting of Symdeko, Kalydeco, Orkambi, and combinations thereof.

The term “TRIKAFTA” or “KAFTRIO,” is the brand name for the combination of elexacaftor, tezacaftor, and ivacaftor. TRIKAFTA is a prescription medicine used for the treatment of cystic fibrosis (CF) in patients who have at least one copy of the F508del mutation in the cystic fibrosis transmembrane conductance regulator (CFTR) gene or another mutation that is responsive to treatment with TRIKAFTA.

The term “SYMDEKO” is the brand name for the combination of tezacaftor and ivacaftor. SYMDEKO is used for treatment of patients with cystic fibrosis (CF) who are homozygous for the F508del mutation or who have at least one mutation in the cystic fibrosis transmembrane conductance regulator (CFTR) gene that is responsive to tezacaftor/ivacaftor based on in vitro data and/or clinical evidence.

The term “KALYDECO” is the brand name for ivacaftor, which has a chemical formula C₂₄H₂₈N₂O₃ and a chemical name N-(2,4-ditert-butyl-5-hydroxyphenl)-4-oxo-1H-quinoline-3-carboxamide.

The term “ORKAMBI” is the brand name for the combination of lumacaftor and ivacaftor that is used to treat people with cystic fibrosis who have two copies of the F508del mutation.

Disclosed also include a method of treating CF characterized by a class I nonsense mutation in the cystic fibrosis transmembrane conductance regulator (CFTR) gene in a subject in need thereof. The method may comprise administering to the subject an effective amount of a SGLT/Na⁺/K⁺-ATPase dual inhibitor and an effective amount of a Na⁺/K⁺-ATPase inhibitor.

In some embodiments, the effective amount of the Na⁺/K⁺-ATPase inhibitor is a subeffective amount.

In some embodiments, the SGLT/Na⁺/K⁺-ATPase dual inhibitor is selected from the group consisting of phlorizin, T-1095, licogliflozin, trilobatin, sergliflozin, remogliflozin, ipragliflozin, luseogliflozin, tofogliflozin, empagliflozin, canagliflozin, and analogs thereof.

In some embodiments, the Na⁺/K⁺-ATPase inhibitor is selected from the group consisting of pyruvic acid, acevaltrate, ouabain, bufalin, istaroxime, biacetyl monoxime, rostafuroxin, gitoxin, oleandrin, deslanoside, chlorpropamide, periplocin, digoxin, rostafuroxin, and analogs thereof. In some embodiments, the Na⁺/K⁺-ATPase inhibitor is ouabain.

In some embodiments, the class I nonsense mutation is a G542X mutation. In some embodiments, the class I nonsense mutation is a W1282X mutation.

In some embodiments, the inhibitors and/or the therapeutic agents utilized in the methods disclosed herein may be formulated as a pharmaceutical composition. The pharmaceutical composition may further comprise one or more pharmaceutically acceptable carrier, excipient, or diluent. As one skilled in the art will also appreciate, the disclosed pharmaceutical compositions can be prepared with materials (e.g., actives excipients, carriers, and diluents etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The inhibitors and/or the therapeutic agents utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. Alternatively, the inhibitors utilized in the methods disclosed herein may be formulated as a pharmaceutical composition in liquid form (e.g., an injectable liquid or gel)

The inhibitors and/or the therapeutic agents utilized in the methods disclosed herein also may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents for the pharmaceutical compositions may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

In some embodiments, the pharmaceutical compositions as described herein can be administered in combination with adjuvants that enhance stability of the agents, facilitate administration of pharmaceutical compositions containing them in certain embodiments, provide increased dissolution or dispersion, increase activity, provide adjuvant therapy, and the like, including other active ingredients.

Pharmaceutical compositions comprising the compounds may be adapted for administration by any appropriate route, for example by the oral (including buccal or sublingual), nasal, inhalation, parenteral (including subcutaneous, intramuscular, intravenous or intradermal) route, or direct injection or administration to a tumor. Such formulations may be prepared by any method known in the art of pharmacy, for example by bringing into association the active ingredient with the carrier(s) or excipient(s).

Pharmaceutical compositions adapted for oral administration may be presented as discrete units such as capsules, pills, tablets, powders, granules, dragees, liquids, gels, syrups; slurries, solutions, or suspensions in aqueous or non-aqueous liquids; edible foams or whips; or oil-in-water liquid emulsions or water-in-oil liquid emulsions.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators. For nasal or inhalation delivery, the compositions of the disclosure also may be formulated by methods known to those of skill in the art, and may include, for example, sprays, inhalers, vapors; solubilizing, diluting, or dispersing substances, such as saline, preservatives, such as benzyl alcohol; absorption promoters; and fluorocarbons may be included.

Pharmaceutical compositions adapted for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

In some embodiments, the composition comprises phlorizin and Trikafta.

In some embodiments, the composition comprises mizagliflozin and Trikafta.

In some embodiments, the composition comprises phlorizin and ouabain.

In some embodiments, the class I nonsense mutation is selected from the group consisting of a G542X mutation, a W1282X mutation, and combinations thereof.

In some embodiments, the CF is characterized by a class II dF508 mutation.

### Examples

Cystic fibrosis (CF) is a lethal autosomal recessive inherited disease characterized by obstructive pulmonary disease, caused by mutations in the cystic fibrosis transmembrane conductance regulator (CFTR) gene (Stoltz D A, et al., 2015). On Oct. 21, 2019, the Food and Drug Administration (FDA) approved TRIKAFTA, which is a highly effective modulator therapy (Collins, F S (2019), provides benefits to 12 years and older with at least one F508del mutation, which affects 90% of the population with cystic fibrosis in the United States. TRIKAFTA (elexacaftor/tezacaftor/ivacaftor) treats 90% of the cystic fibrosis (CF) population with the most common CF transmembrane conductance regulator (CFTR) mutations including Class II, III, IV, and VI mutations. However, TRIKAFTA causes serious adverse reactions including hepatic injury and hypersensitivity. Further, TRIKAFTA does not treat Class I mutations.

Recently, it was reported that Wnt signaling regulates lung differentiation of PSCs and that low-Wnt conditions allowed derivation of human proximal lung organoids (HLOs) from purified PSC-derived lung epithelial cells. These lung organoids, when subjected to a forskolin (Fsk)-stimulated swelling assay, respond in a mutation-dependent manner: normal CFTR wild-type (WT) organoids swell rapidly, whereas CF organoids show minimal expansion of size. The swelling assay hence represents a simple and robust method to measure CFTR function in the organoids (McCauley K B, et al., 2017, Hirai H, et al., 2022).

The Sodium-dependent glucose cotransporters 1 (SGLT1) belong to a human gene family, SLC5A1 (Sano R, et al., 2020). SGLT1 is expressed in many tissues, including CF-relevant ones such as lung, intestine and trachea (Wright E M, et al., 2011, Gagnon K B, et al., 2021). It was demonstrated that SGLT1 inhibitors Phlorizin and Sotagliflozin can markedly restore the CF swelling function of the HLOs with class II mutations (Hirai H, et al., 2022). However, the effects of combination of these inhibitors with TRIKAFTA on HLOs with Class II mutations are not studied.

HLOs were derived using CFTR iPSCs with either Class I or II mutations and it was discovered that combination of low dose of TRIKAFTA and SGLT1 inhibitor Mizagliflozin or combination of low dose of TRIKAFTA and SGLT1/Na⁺/K⁺-ATPase dual inhibitor Phlorizin are therapies for CF with Class II mutations with potentially better safety profile. In addition, the combination of Na⁺/K⁺-ATPase inhibitor and SGLT/Na⁺/K⁺-ATPase dual inhibitor may be therapeutic agents for CF with Class I mutations.

Derivation of HLOs Using iPSCs Carrying CFTR Class I and II Mutations

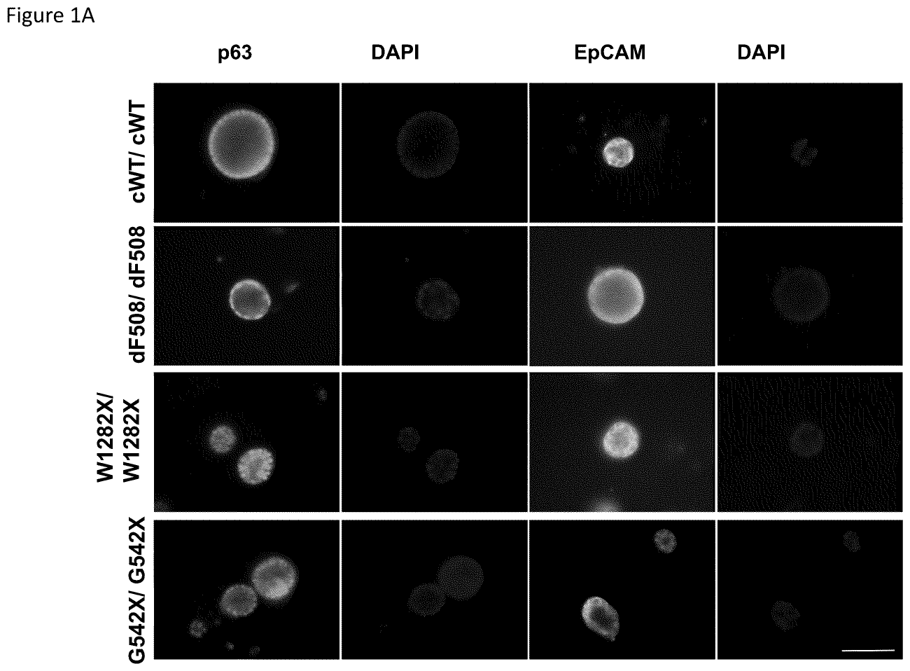

iPSCs carrying the homozygous CFTR delF508 (dF508/dF508) mutation (class II mutation) (ATCC; Manassas, VA), homozygous CPTR nonsense mutations W1282X (class I mutation) (Cystic Fibrosis Foundation; Bethesda, MD) G542X (class I mutation) (Cystic Fibrosis Foundation; Bethesda, MD) and a homozygously corrected wild-type/wild-type (cWT/cWT) (Hirai et al, 2022) were successfully differentiated into proximate lung lineage cells, following a 22-day regime as previously described (Hirai et al, 2022). Both dF508/dF508 and W1282X/W1282X HLOs were positive for p63 and EpCAM, demonstrating the derivation HLOs carrying CFTR class I and II mutations (FIG. 1A).

### ATP1A1 and SGLT1 is Upregulated in CF HLOs

Western blotting showed that the ATP1A1 and SGLT1 expression were markedly increased in HLOs carrying either CFTR class I or II mutations than those from non-CF controls, cWT/cWT (FIG. 1B).

SGLT1 Inhibitor Mizagliflozin Restores HLO Swelling of Class II CFTR Mutations in Combination with 125-Fold Diluted TRIKAFTA

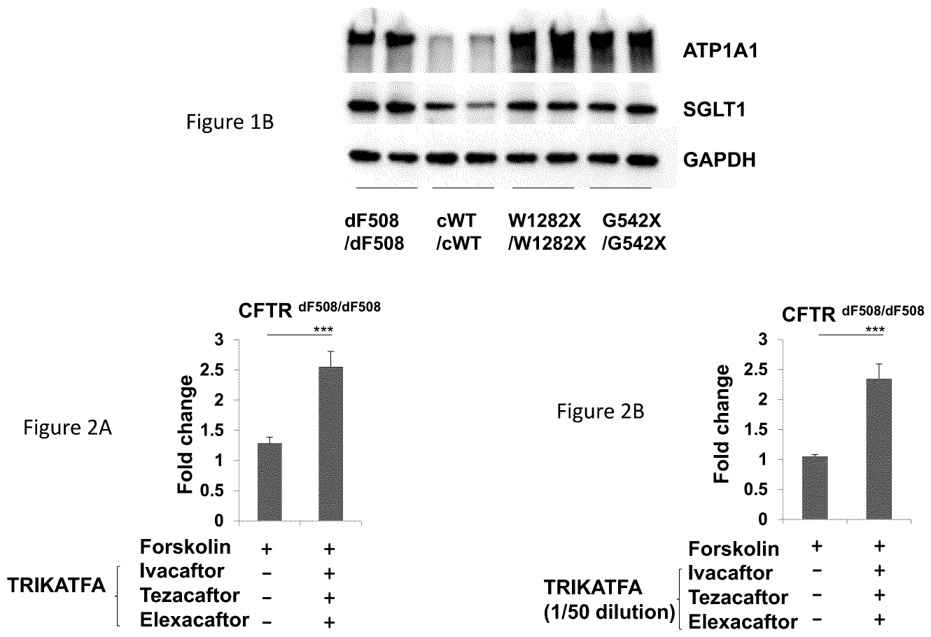

The dF508/dF508 HLOs were subjected to fsk-stimulated swelling assay. As shown in FIGS. 2A and 2B, 50 or 125-fold diluted TRIKAFTA increased the size of the HLOs less effectively compared to undiluted TRIKAFTA. However, Mizagliflozin combination with 125-fold diluted TRIKAFTA had recovered to 97% of original dF508/dF508 swelling level which was almost same size level induced by undiluted TRIKAFTA (FIGS. 2A and 2C), supporting the synergistic effect of TRIKAFTA and Mizagliflozin and its analogs.

Combination of Low Dose of TRIKAFTA and SGLT/Na⁺/K⁺-ATPase Dual Inhibitor Phlorizin Restores HLO Swelling of Class II CFTR dF508/dF508 Mutation.

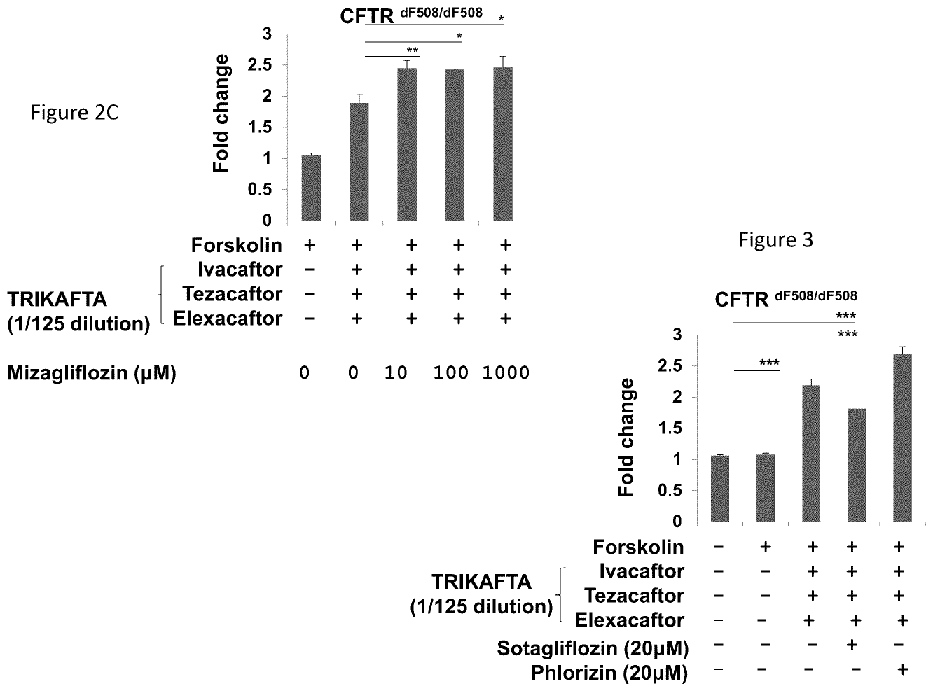

Phlorizin is a SGLT and Na⁺/K⁺-ATPase dual inhibitor (Nakagawa A, et al., 1977). Next, studies were carried out to determine if SGLT and Na⁺/K⁺-ATPase dual inhibition would lead to restoration of swelling function of HLOs with Class II mutations with 125-fold diluted TRIKAFTA. Surprisingly, 125-fold diluted TRIKAFTA combined with Phlorizin but not Sotagliflozin (a SGLT2-selective inhibitor) had completely recovered dF508/dF508 swelling size, and even more than 10% promoted the swelling size compared to undiluted TRIKAFTA treatment (FIG. 3), strongly supporting the potentially better safety profile of combination therapy of low TRIKAFTA dose with Phlorizin.

Na⁺/K⁺-ATPase Inhibitors Pyruvic Acid and Acevaltrate Restore HLO Swelling of Class I CFTR Mutations

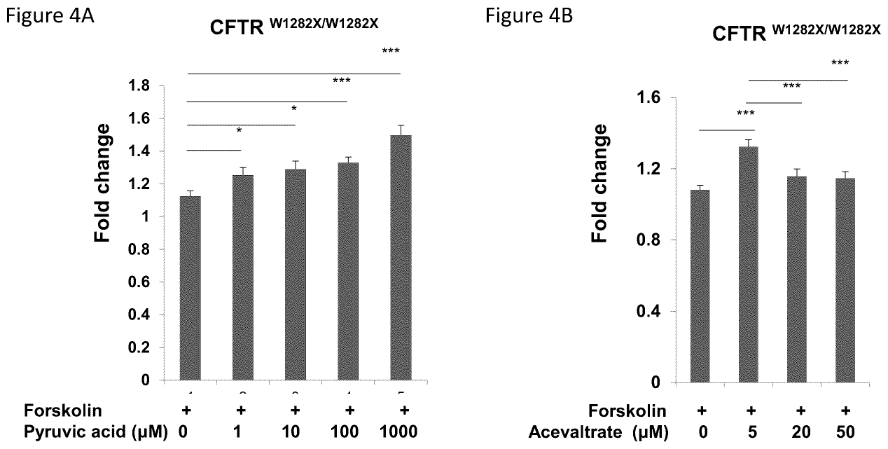

Next, two Na⁺/K⁺-ATPase inhibitors pyruvic acid and Acevaltrate were employed to demonstrate additional drugs for therapy of CF with Class I mutations. Consistently, pyruvic acid increased the size of W1282X/W1282X HLOs swelling by 33% with 1000 μM concentration, compared to foskolin-stimulated (fsk-stimulated) swelling (FIG. 4A). The effects of another Na⁺/K⁺-ATPase inhibitor Acevaltrate was also assessed. Acevaltrate treatment also increased the size of W1282X/W1282X HLOs swelling by 22% at 5 μM concentration (FIG. 4B). Together, these data demonstrate the therapeutic potential of Na⁺/K⁺-ATPase inhibitors and their analogs for treating CF with Class I mutation.

Combination of Na⁺/K-ATPase Inhibitor Ouabain with SGLT/Na*/K-ATPase Dual Inhibitor Phlorizin Restores HLO Swelling of Class I CFTR Mutation

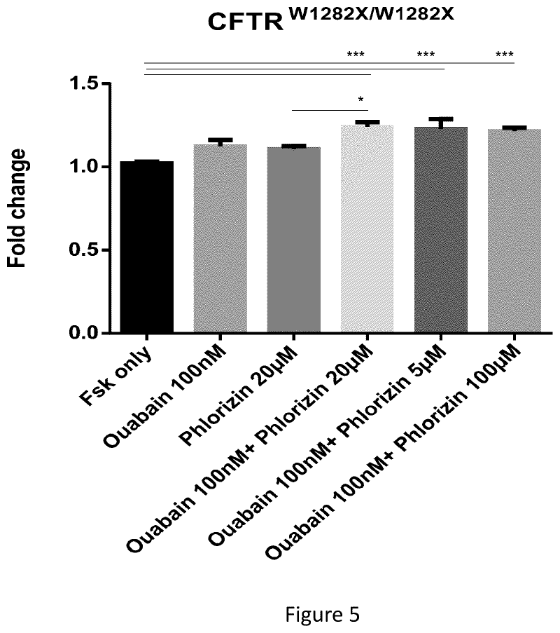

Next, Na⁺/K⁺-ATPase inhibitor Ouabain and SGLT/Na⁺/K⁺-ATPase dual inhibitor Phlorizin were employed to demonstrate the combination effect of drug treatment for therapy of HLO of CF with Class I mutations. Ouabain or Phlorizin alone only increased the swelling size of W1282X/W1282X HLOs by 11%. Surprisingly, combination of 100 nM Ouabain and 5, 20, or 100 μM of Phlorizin increased the size of W1282X/W1282X HLOs swelling by 22% compared to fsk-stimulated swelling (FIG. 5).

In summary, the isogenic human proximal lung organoids (HLOs) from gene-edited patient-derived pluripotent stem cells (PSCs) carrying different CFTR mutations, and the Forskolin-stimulated HLO swelling assay, a measurement of CFTR function, were employed and it was discovered that the Mizagliflozin, a sodium/glucose cotransporter 1(SGLT1) inhibitor synergistically restored the defective swelling function of forskolin-stimulated CF HLOs derived from PSCs harboring class II CFTR mutations dF508/dF508 with 125-fold reduced dose of TRIKAFTA. Surprisingly, combination of SGLT/Na⁺/K⁺-ATPase dual inhibitor, Phlorizin with 125-fold reduced dose of TRIKAFTA, synergistically recovered the defect of forskolin-stimulated HLO swelling of CF HLOs derived from PSCs harboring class II CFTR mutations dF508/dF508. Together, it was shown that combination of low dose of TRIKAFTA and SGLT1 inhibitor or the combination of TRIKAFTA and SGLT/Na⁺/K⁺ dual inhibitor is a safer and effective therapeutic treatment for CF. Thus, SGLT1 inhibitor Mizagliflozin, and their analogs, or SGLT/Na⁺/K⁺ dual inhibitor phlorizin and their analogs thereof, when combined with low dose of TRIKAFTA, has great potential for treatment of Cystic Fibrosis with a better safety profile.

Further, Na⁺/K⁺-ATPase inhibitors pyruvic acid and Acevaltrate restored swelling defect of HLOs harboring class I CFTR mutations. Furthermore, combination of Na⁺/K⁺-ATPase inhibitor Ouabain with SGLT/Na⁺/K⁺-ATPase dual inhibitor Phlorizin effectively restored swelling defect of aged HLOs harboring class I CFTR mutations W1282X/W1282X. Thus, the combination of Na⁺/K⁺-ATPase inhibitors and SGLT/Na⁺/K⁺-ATPase dual inhibitors, and their analogs have great potential for treatment of Cystic Fibrosis, especially class I mutations.

Differentiation of Definitive Endoderm Cells from iPSCs
iPSCs were harvested and dissociated into a single-cell suspension with using Gentle Cell Dissociation Reagent (Stem Cell Technologies, Vancouver, Canada) and seeded onto Corning Matrigel hESC-qualified Matrix (Corning, Corning, NY) coated plate (Corning) in mTesR1 (Stem Cell Technologies) containing 10 μM Y-27632 (Stem Cell Technologies) for 24 hr. Then iPSCs were differentiated into definitive endoderm using STEMdiff Definitive Endoderm Kit (Stem Cell Technologies) for 72 hr.
   Differentiation of Anterior Foregut Endoderm Cells from Definitive Endoderm Cells

Definitive endoderm cells were treated for 72 hr with anterior foregut endoderm differentiation medium containing Ham's F-12 Nutrient Mix (Thermo Fisher Scientific Waltham, MA, USA) and IMDM (Thermo Fisher Scientific, Waltham, MA) with B27 Supplement (Thermo Fisher Scientific), N2 Supplement (Thermo Fisher Scientific), 0.1% Bovine Serum Albumin Fraction V (Sigma-Aldrich, St. Louis, MO), 1-Thioglycerol (Sigma-Aldrich), 1×GlutaMAX Supplement (Thermo Fisher Scientific), and 1% penicillin-streptomycin, 50 μg/ml L-ascorbic acid, 10 mM SB431542 (Cayman Chemical, Ann Arbor, MT) and 2 mM Dorsomorphin (Cayman Chemical).

Differentiation of Lung Epithelial Progenitors from Anterior Foregut Endoderm Cells

Anterior foregut endoderm cells were then treated for 8 days with lung epithelial progenitor differentiation medium containing Ham's F-12 Nutrient Mix and IMDM with B27 Supplement, N2 Supplement, 0.1% Bovine Serum Albumin Fraction V, 1-Thioglycerol, 1× GlutaMAX Supplement, and 1% penicillin-streptomycin, and 1Ong/ml Human Recombinant BMP4 (Stem Cell Technologies), 50 μg/ml L-ascorbic acid, 3 mM CHIR99021 (Cayman Chemical), and 100 nM Retinoic acid (Sigma-Aldrich).

Differentiation of Proximal Lung Organoids from Lung Epithelial Progenitors

On day 14-15 lung epithelial progenitors were dissociated into single cell suspensions with Trypsin-EDTA (0.05%) (Thermo Fisher Scientific). Harvested cells are immunostained with CD47 and CD26 antibodies. CD47+high/positive and CD26-low/negative cells (CD47hi/CD261o) were sorted, followed by resuspending as single cells in 50 μl three-dimensional growth factor reduced Matrigel drops, treated with proximal lung organoid differentiation medium containing Ham's F-12 Nutrient Mix and IMDM with B27 Supplement, N2 Supplement, 0.1% Bovine Serum Albumin Fraction V, 1-Thioglycerol, 1×GlutaMAX Supplement, and 1% penicillin-streptomycin, 100 ng/ml Human Recombinant FGF10, 250 ng/ml Human Recombinant bFGF (Stem Cell Technologies), 50 μg/ml L-ascorbic acid, 100 nM Dexamethasone (Cayman Chemical), 0.1 mM 8-bromo-Cyclic AMP (8-bromo-cAMP) (Cayman Chemical), and 10 mM 3-isobutyl-1-methylxanthine (Sigma-Aldrich) and Y-27632 (Cayman Chemical).

### Forskolin-Induced Swelling in Proximal Lung Organoid Organoids

CFTR function was quantified by measuring fsk-induced swelling of organoids as described previously (Hirai H, et al., 2022). Organoids were incubated with or without 10 μM fsk (Selleck Chemicals, Houston, TX, USA), and swelling was monitored using time-lapse microscopy. To evaluate the effects of different compounds on the swelling, Mizagliflozin (Selleck Chemicals), Phlorizin (Selleck Chemicals), Pyruvic acid (Selleck Chemicals), Acevaltrate (MedChemExpress, Monmouth Junction, NJ, USA), Chlorpropamide (Selleck Chemicals), Ouabain (Bio-Techne Corporation, Minneapolis, MN, USA), Elexacaftor (Selleck Chemicals) Tezacaftor (Cayman Chemical) or Ivacaftor (Selleck Chemicals) was added to the culture medium according to the experimental design 24 h before fsk treatment.

### Immunofluorescence Staining

Fixed cultured cells with 4% Paraformaldehyde (PFA) in PBS for 10 min and permeabilized with 0.5% Triton X-100 (Sigma-Aldrich) in PBS for 5 min at room temperature. Cells were stained with the primary antibody TP63 (1:100, CM163A, Biocare Medical) or PE-conjugated human EPCAM (I:200, 12-9326-42, Thermo Fisher Scientific) for 1 hr and goat anti-Mouse IgG (H+L) Highly Cross-Adsorbed Secondary Antibody Alexa Fluor 488 conjugate (I:200, Al I 029; Thermo Fisher Scientific) for 45 min at room temperature. Nuclei were counterstained with DAPI.

### Western Blot Analysis

Equal amounts of protein lysates from cultured organoids were resolved by SDS-PAGE and transferred to polyvinylidene difluoride (PVDF) membranes (PerkinElmer, Boston, MA, USA), which then were blocked at room temperature for 1 h with 5% (w/v) skim milk powder in TBST (10 mM Tris, pH 8.0, 150 mM NaCl, and 0.05% Tween 20). The blots were incubated with primary antibodies: anti-ATP1A1 (catalog #14418-1-AP; Proteintech, Rosemont, IL, USA) at 1:10,000, anti-SGLT1 (catalog #5042; Cell Signaling Technology, Danvers, MA, USA) at 1:1,000, or anti-GAPDH Polyclonal antibody (catalog #10494-1-AP; Proteintech) at 1:10,000 for overnight at 4° C. on a rotating shaker. After washing with TBST for 10 min and three times each with TBST, the blots were then incubated with horseradish peroxidase-conjugated secondary antibody for 1 h at 4° C. on a rotating shaker. Western blots were scanned and quantified using an iBrigh FL1500 Imaging System (Thermo Fisher Scientific).

### Statistical Analysis

Data are presented as mean±SEM. Student's t-test (2-tailed) was used to compare data using GraphPad Prism 8 software (GraphPad Software, Inc., San Diego, CA). P values<0.05 were considered statistically significant. ***p<0.001, **p<0.005, *p<0.05.

### REFERENCES

Bear C. (2020). A Therapy for Most with Cystic Fibrosis. Cell 180, 211.
Collins, F S (2019). Realizing the dream of molecularly targeted therapies for cystic fibrosis. N Engl. J Med. 381, 1863-1865.
Gagnon K B, Delpire E. (2021). Sodium Transporters in Human Health and Disease. Front Physiol. 11, 588664.
Hirai H, Liang X, Sun Y, Zhang Y, Zhang J, Chen Y E, Mou H, Zhao Y, Xu J. (2022). The sodium/glucose cotransporters as potential therapeutic targets for CF lung diseases revealed by human lung organoid swelling assay. Mol Ther Methods Clin Dev. 24, 11-19.
McCauley K B, Hawkins F, Serra M, Thomas D C, Jacob A, Kotton D N. (2017). Efficient derivation of functional human airway epithelium from pluripotent stem cells via temporal regulation of Wnt signaling. Cell Stem Cell 20, 844-857.
Nakagawa A, and Nakao M. (1977). Localization of the phlorizin site on Na, K-ATPase in red cell membranes. J Biochem. 81, 1511-1515.
Sano R, Shinozaki Y, Ohta T. (2020). Sodium-glucose cotransporters: functional properties and pharmaceutical potential. J Diabetes Invest. 11, 770-782.
Stoltz D A, Meyerholz D K, Welsh M J. (2015). Origins of cystic fibrosis lung disease. N Engl. J Med. 372, 1574-1575.
Wright E M, Hirayama B A. (2011). Biology of human sodium glucose transporters. Physiol Rev. 91, 733-94.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating cystic fibrosis (CF) comprising administering to a subject an effective amount of an inhibitor selected from the group consisting of a sodium glucose co-transporter (SGLT) inhibitor, a Na⁺/K⁺-ATPase inhibitor, a SGLT/Na⁺/K⁺-ATPase dual inhibitor, and combinations thereof and a subeffective amount of one or more therapeutic agents selected from the group consisting of elexacaftor, tezacaftor, ivacaftor, and combinations thereof.

2. The method of claim 1, wherein CF is characterized by a mutation in the cystic fibrosis transmembrane conductance regulator (CFTR) gene selected from the group consisting of class I mutation, class II mutation, class III mutation, class IV mutation, and class VI mutation.

3. The method of claim 1, wherein CF is characterized by a class I mutation in the cystic fibrosis transmembrane conductance regulator (CFTR) gene.

4. The method of claim 1, wherein CF is characterized by a class II mutation in the cystic fibrosis transmembrane conductance regulator (CFTR) gene.

5. The method of claim 1, wherein the inhibitor is the SGLT/Na⁺/K⁺-ATPase dual inhibitor.

6. The method of claim 5, wherein the SGLT/Na⁺/K⁺-ATPase dual inhibitor is selected from the group consisting of phlorizin, trilobatin, T-1095, licogliflozin, trilobatin, sergliflozin, remogliflozin, ipragliflozin, luseogliflozin, tofogliflozin, empagliflozin, canagliflozin, and dapagliflozin, and analogs thereof.

7. The method of claim 1, wherein the inhibitor is a SGLT inhibitor.

8. The method of claim 7, wherein the inhibitor is a SGLT1-selective inhibitor.

9. The method of claim 8, wherein the SGLT1-selective inhibitor is selected from the group consisting of KGA-2727, LX2761, LX276123, TP043883625, SGL5213, SGLT inhibitor 1, mizagliflozin, and analogs thereof.

10. The method of claim 9, wherein the SGLT1-selective inhibitor is mizagliflozin or analogs thereof.

11. The method of claim 7, wherein the inhibitor is a non-selective SGLT inhibitor.

12. The method of claim 11, wherein the non-selective SGLT inhibitor is phloretin or analogs thereof.

13. The method of claim 1, wherein the inhibitor is a Na⁺/K⁺-ATPase inhibitor.

14. The method of claim 11, wherein the Na⁺/K⁺-ATPase inhibitor is selected from the group consisting of pyruvic acid, acevaltrate, ouabain, bufalin, istaroxime, biacetyl monoxime, rostafuroxin, gitoxin, oleandrin, deslanoside, chlorpropamide, periplocin, digoxin, rostafuroxin, and analogs thereof.

15. The method of claim 1, wherein the method comprises administering a subeffective amount of elexacaftor, tezacaftor, and ivacaftor.

16. A method for treating cystic fibrosis (CF) characterized by a class I mutation in the cystic fibrosis transmembrane conductance regulator (CFTR) gene, the method comprising administering to a subject an effective amount of a Na⁺/K⁺-ATPase inhibitors selected from the group consisting of pyruvic acid, acevaltrate, analogs thereof, and combinations thereof.

17. A method for treating cystic fibrosis (CF) characterized by a class I nonsense mutation in the cystic fibrosis transmembrane conductance regulator (CFTR) gene in a subject in need thereof, the method comprising administering to the subject an effective amount of a SGLT/Na⁺/K⁺-ATPase dual inhibitor and an effective amount of a Na⁺/K⁺-ATPase inhibitor.

18. The method of claim 17, wherein the effective amount of the Na⁺/K⁺-ATPase inhibitor is a subeffective amount.

19. The method of claim 17, wherein the SGLT/Na⁺/K⁺-ATPase dual inhibitor is selected from the group consisting of phlorizin, T-1095, licogliflozin, trilobatin, sergliflozin, remogliflozin, ipragliflozin, luseogliflozin, tofogliflozin, empagliflozin, canagliflozin, dapagliflozin, and analogs thereof.

20. The method of claim 17, wherein the Na⁺/K⁺-ATPase inhibitor is selected from the group consisting of pyruvic acid, acevaltrate, ouabain, bufalin, istaroxime, biacetyl monoxime, rostafuroxin, gitoxin, oleandrin, deslanoside, chlorpropamide, periplocin, digoxin, rostafuroxin, and analogs thereof.

21. The method of claim 17, wherein the class I nonsense mutation is a G542X mutation or a W1282X mutation.
